(19) United States
(12) Patent Application Publication
(10) Pub. No.: US 2022/0397565 A1
(43) Pub. Date: Dec. 15, 2022

(54) METHODS OF SCREENING AND RELATED SYSTEMS

(71) Applicant: RECURSION PHARMACEUTICALS, INC., Salt Lake City, UT (US)

(72) Inventors: Chris Johnson, Salt Lake City, UT (US); Ian Quigley, Salt Lake City, UT (US); Renat Khaliullin, Salt Lake City, UT (US); Timothy Dahlem, Salt Lake City, UT (US); Daniel Anderson, Salt Lake City, UT (US)

(21) Appl. No.: 17/344,593

(22) Filed: Jun. 10, 2021

(51) Int. Cl. G01N 33/50; C12N 15/10; G01N 1/30; G01N 15/14; G16H 70/40

(52) U.S. Cl. CPC G01N 33/5026; G01N 33/5023; C12N 15/1072; G01N 1/30; G01N 15/1475; G16H 70/40; G01N 2015/1472; G01N 2015/1497

(57) ABSTRACT

Disclosed herein are methods of high-throughput screening test agents for treating a disease. Also disclosed herein are methods of high-throughput screening diseases. Systems for high-throughput screening are also disclosed herein.

### COPYRIGHT NOTICE

© 2021 Recursion Pharmaceuticals, Inc. A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR § 1.71(d).

### TECHNICAL FIELD

This disclosure relates generally to methods of screening and related systems, and in particular the disclosure relates to methods of screening involving gain of function mutations and related systems.

### BRIEF DESCRIPTION OF THE DRAWINGS

The embodiments disclosed herein will become more fully apparent from the following description and appended claims, taken in conjunction with the accompanying drawings. The drawings depict primarily generalized embodiments, which embodiments will be described with additional specificity and detail in connection with the drawings in which:

FIG. 1 is a fluorescent microscopy image of BRAF V600E transduced cells (left panel) and BRAF V600E nonsense transduced cells (right panel) from Example 1.

FIG. 2 is a heatmap of cosine similarity between pairs of vectors of image-based features extracted from images of cells treated by the reagents in the BacMam titration experiment of Example 1.

FIG. 3 is a zoom-in image of FIG. 2.

FIG. 4A is a fluorescent microscopy image of INS-1E cells (left panel) and INS-1E cells overexpressing wildtype MFN1 (right panel) from Park et al. Korean J Physiol Pharmacol. 2012 Feb.; 16(1): 71-77.

FIG. 4B is a fluorescent microscopy image of U2OS cells (left panels) and U2OS cells overexpressing wildtype MFN1 (right panels) from Example 2.

FIG. 5 are microscopy images of KB cells from Bretscher et al. Current Biology Volume 8, Issue 12, 4 Jun. 1998, Pages 721-724, S1-S4 and U2OS cells transduced to overexpress EGFR FLAG from Example 2. Unstimulated KB cells (first left panel) and KB cells stimulated with EGF (second panel from the left) are shown. Microscopy images of the U2OS cells for FLAG (far right), nucleus (second from right), and merged FLAG and nucleus (center) are shown.

FIG. 6 shows fluorescent microscopy images of hairy cell leukemia samples stained with Draq5, Phalloidin, and Annexin V (first left panel, image from Falini et al. Blood. 2016;128(15):1918-1927, and BRAF V600E FLAG overexpressing U2OS cells showing FLAG (far right), nucleus (second from right), and merged FLAG and nucleus (third from right) from Example 2.

FIG. 7 is a heatmap of cosine similarities between all pairs of reagent groups.

FIG. 8 is a heatmap of cosine similarities between all pairs of feature vectors that were origined on samples within the block of similarity described in FIG. 7.

FIG. 9 is a heatmap of cosine similarities comparing cells expressing EGFR FLAG with various test agents from Example 3.

FIG. 10 is a dot plot of side effect score versus disease score of cells expressing BRAF V600E treated with ulixertinib, dabrafenib, MEK162, and vemurafenib from Example 4.

FIG. 11 is a block diagram of a high-throughput screening system in accordance with one embodiment. To easily identify the discussion of any particular element or act, the most significant digit or digits in a reference number refer to the figure number in which that element is first introduced.

### DETAILED DESCRIPTION

Disclosed herein are methods of high-throughput screening test agents for treating a disease. In a first aspect, the methods comprise cultivating pathogenic transduced cells overexpressing a gene of interest associated with a disease to exhibit a disease phenotype in cultivated pathogenic transduced cells and cultivating non-pathogenic transduced cells not overexpressing the gene of interest to exhibit a control phenotype in cultivated non-pathogenic transduced cells. The methods further comprise profiling the cultivated pathogenic transduced cells to develop a disease phenotype profile and profiling the cultivated non-pathogenic transduced cells to develop a control phenotype profile. The methods further include adding one or more of a plurality of test agents, or combinations thereof, to the cultivated pathogenic transduced cells to form treated cultivated pathogenic transduced cells. Additionally, the methods include profiling the treated cultivated pathogenic transduced cells and identifying any of the plurality of the test agents, or combinations thereof, that modulate the disease phenotype profile of the treated cultivated pathogenic transduced cells to a treated profile that is similar to that of the control phenotype profile. The test agents used in the method may include small molecules, biological compounds, or combinations thereof.

In various embodiments, the phenotypic profile may be generated from a profiling process include metabolomic profiling, proteomic profiling, gene expression profiling, genetic profiling, morphological profiling, or combinations thereof. For example, for morphological profiling, the methods may comprise cultivating pathogenic transduced cells overexpressing a gene of interest associated with a disease to exhibit a disease phenotype in cultivated pathogenic transduced cells and cultivating non-pathogenic transduced cells not overexpressing the gene of interest to exhibit a control phenotype in cultivated non-pathogenic transduced cells. The methods further comprise morphologically profiling the cultivated pathogenic transduced cells to develop a disease phenotype morphological profile and morphologically profiling the cultivated non-pathogenic transduced cells to develop a control phenotype morphological profile. The methods further include adding one or more of a plurality of test agents, or combinations thereof, to the cultivated pathogenic transduced cells to form treated cultivated pathogenic transduced cells. Additionally, the methods include morphologically profiling the treated cultivated pathogenic transduced cells and identifying any of the plurality of the test agents, or combinations thereof, that modulate the disease phenotype morphological profile of the treated cultivated pathogenic transduced cells to a treated morphological profile that is similar to that of the control phenotype morphological profile. The test agents used in the method may include small molecules, biological compounds, or combinations thereof.

Morphological profiles may be generated by various methods. In an embodiment, morphologically profiling cells may comprise imaging relevant cells or tracking certain parameters or features of the cells. The tracking may be of staining intensities in one or more imaging channels, correlations between imaging channels, textural patterns, size and shape of cellular structures, geometric relationships between intracellular structures, geometric relationships between adjacent cells, or combinations thereof. Cellular features, such as 100 or more cellular features, 500 or more cellular features, or 1000 or more cellular features, may be tracked when morphologically profiling cells. Among various methods of determining these cellular features, they may be obtained by imaging cells with or without fluorescent or non-fluorescent stains or combinations thereof, and numeric features determined by using deep learning embeddings, hand-tuned feature extraction, or combinations thereof. Other than tracking the cellular features, the morphological profiling may include reducing the output of the cellular features using statistical modeling such as principal component analysis.

Cells that overexpress a gene of interest, for example those that are associated with a gain-of-function disease, and cells that do not overexpress the gene of interest may be used in the method of the first aspect. In an embodiment, the method of the first aspect can further comprise prior to the cultivating steps obtaining pathogenic transduced cells overexpressing a gene of interest associated with a gain-of-function disease and obtaining non-pathogenic transduced cells not overexpressing the gene of interest.

Other profiles may be obtained that are not morphological in nature. A variety of technologies can be used to measure molecular features in a biased or unbiased way at scale. These include, but are not limited to, transcriptional profiling (e.g. RNA-seq), chromatin accessibility or chromatin state (e.g. ATAC-seq), 3D genome structure (e.g. Hi-C), metabolomics, or proteomics. Like imaging features, these high-dimensional measurements can be reduced to a smaller number of features using statistical modeling such as principal component analysis.

Genes of interest may be introduced to cells by various methods, such as by using viral vectors or Bacmam vectors. In an embodiment, the method of the first aspect obtains pathogenic transduced cells overexpressing the gene of interest by introducing Bacmam vectors that contain the gene of interest and are configured to be transduced into cells to cause overexpression of the gene of interest. Likewise, obtaining non-pathogenic transduced cells not overexpressing the gene of interest comprises introducing Bacmam vectors not configured to overexpress the gene of interest. The Bacmam vectors not configured to overexpress the gene of interest may have: no genetic payload; carry nucleic acids encoding for a scrambled messenger ribonucleic acid; carry the gene of interest but configured to localize the encoded protein incorrectly or to express a truncated protein; carry a non-pathogenic gene for overexpression; or combinations thereof.

An additional approach to perturbing gene expression with Bacmam vectors would be to have the vector encode a Cas9 variant. In this embodiment, Cas9, or Cas9 fused with a functional domain (including but not restricted to a transcriptionally transactivating domain, this construct is colloquially referred to as CRISPRa, or an inhibiting domain, referred to as CRISPRi), is introduced to the cells with a Bacmam vector, and then guide RNAs are introduced by other means (e.g. lipofection) in order to direct the Cas9 proteins to desired genomic targets. This approach would allow for the overexpression or repression of any gene product but would only require the construction of a single Bacmam vector.

Bacmam vector concentrations used to obtain pathogenic transduced cells and the concentrations used to obtain non-pathogenic transduced cells may be equal. Alternatively, Bacmam vector concentrations used to obtain pathogenic transduced cells and the vector concentrations used to obtain non-pathogenic transduced cells may be different.

The method of the first aspect can further comprise cultivating pathogenic transduced cells that overexpress different genes of interest to exhibit different disease phenotypes in each reservoir or well of a cell culture vessel or plate. In an embodiment, the gene of interest comprises a first gene of interest and wherein cultivating the pathogenic transduced cells to exhibit a disease phenotype in cultivated pathogenic transduced cells comprises cultivating a first pathogenic transduced cells to exhibit a first disease phenotype in a first cultivated pathogenic transduced cells grown in a first reservoir of a cell culture vessel. The gene of interest may also comprise a second gene of interest and cultivating the pathogenic transduced cells to exhibit a disease phenotype in cultivated pathogenic transduced cells comprises cultivating a second pathogenic transduced cells to exhibit a second disease phenotype in a second cultivated pathogenic transduced cells grown in a second well of the well plate.

The first well can contain a first pre-selected initial quantity of cells and the second well can contain a second pre-selected initial quantity of cells. The first pre-selected initial quantity may be different from the second pre-selected initial quantity of cells. After the cultivating step, the quantity of cells in the first well can approximately be equal to the quantity of cells in the second well. In another embodiment, obtaining pathogenic transduced cells overexpressing a gene of interest and obtaining non-pathogenic transduced cells not overexpressing the gene of interest comprises transducing the cells in individual wells of the well plate.

In another embodiment, the cultivated pathogenic transduced cells of the method of the first embodiment also overexpress one or more additional genes associated with the disease, underexpress one or more genes associated with the disease, or combinations thereof.

Similarity can be quantified or scored by using a similarity metric or similarity function. A nonmetric similarity function s(x, x′) is used to compare the two vectors x and x′. Conventionally, s(x, x′) is a symmetric function whose value is large when x and x′ are somehow “similar.” An example of a nonmetric similarity function s(x, x′) is provided on page 216 of Duda, R. O., and P. E. Hart. Pattern Classification and Scene Analysis, 1973, which is incorporated by reference in its entirety herein. For purposes of the present disclosure, the term “test similarity metric” is deemed to encompass similarity metrics, such as the angular distance function of block 442, as well as nonmetric-similarity functions such as those described in Duda, R. O., and P. E. Hart. Pattern Classification and Scene Analysis. 1973.

Scores or values can be determined for each morphological feature or profile to identify the similarity levels between morphological profiles. A morphological profile may be identified as similar to another morphological profile if the determined score is within a range of particular scores. In an embodiment of the first aspect, the methods further comprise determining a score for the treated phenotype morphological profile relative to the control phenotype morphological profile and identifying the treated phenotype morphological profile as similar to the control phenotype morphological profile if the score is within a selected range.

In another embodiment, the methods comprise determining a value for each morphological feature of a specific set of morphological features of the treated morphological profile relative to corresponding morphological features of the control phenotype morphological profile and identifying the treated morphological profile as similar to the control phenotype morphological profile if the value for each morphological feature of the specific set is within a selected range.

In another embodiment, the method of the first aspect further comprises determining a value for each morphological feature of the treated morphological profile relative to corresponding morphological features of the control phenotype morphological profile and identifying the treated morphological profile as similar to the control phenotype morphological profile if a selected percentage of the values are within a selected range.

The selected range in any of the embodiments may be specific to the value for each morphological feature or each of the values may be scaled and the selected range may be a single selected range that applies to all of the scaled values.

Other methods disclosed herein are methods of high-throughput screening diseases. For example, cells from a patient that overexpress a gene associated with a disease, underexpress a gene associated with a disease, or a combination of both may be morphologically profiled and compared with a library of morphological profiles or with a morphological profile of cells from the patient that do not overexpress or underexpress the gene associated with the disease. The methods in a second aspect comprise providing a first disease phenotype morphological profile associated with a first disease and comparing the first disease phenotype morphological profile with a library of a plurality of phenotype morphological profiles, wherein each of the plurality of phenotype morphological profiles were generated from cultivated pathogenic transduced cells that overexpressed one or more genes, underexpressed one or more genes, or combinations thereof. The methods further comprise identifying any of the plurality of the phenotype morphological profiles that are similar to the first disease phenotype morphological profile.

Similar to some embodiments of the method of the first aspect, the methods of the second aspect may further comprise determining scores or values for morphological features or profiles to identify similarity between morphological profiles. In a first embodiment of the method of the second aspect, the methods further comprise determining a score for the first disease phenotype morphological profile relative to each of the plurality of phenotype morphological profiles and identifying the first disease phenotype morphological profile as similar to each of the plurality of phenotype morphological profiles if the score is within a selected range. In a second embodiment of the second aspect, the methods may further comprise determining a value for each morphological feature of a specific set of morphological features of the first disease phenotype morphological profile relative to corresponding morphological features of each of the plurality of phenotype morphological profiles and identifying the first disease phenotype morphological profile as similar to each of the plurality of phenotype morphological profiles if the value for each morphological feature of the specific set is within a selected range. In a third embodiment of the second aspect, the methods further include determining a value for each morphological feature of the first disease phenotype morphological profile relative to corresponding morphological features of each of the plurality of phenotype morphological profiles and identifying the first disease phenotype morphological profile as similar to each of the plurality of phenotype morphological profiles if a selected percentage of the values are within a selected range. The selected ranges may be specific to the value for each morphological feature or each of the values may be scaled and the selected range may be a single selected range that applies to all of the scaled values.

The disease phenotype morphological profiles may comprise morphological profiling of cells from a patient with a disease. In a fourth embodiment of the method of the second aspect, providing the first disease phenotype morphological profile associated with the first disease comprises morphologically profiling cells from a patient having the first disease. Similar to some embodiments of the first aspect, morphologically profiling cells in the embodiments of the second aspect may comprise using cell imaging or tracking various parameters or features of the cells. Also like an embodiment of the first aspect, the morphologically profiling in any of the steps may comprise tracking many cellular features, and the cellular features may be determined and/or the output of the cellular features reduced using any of the methods disclosed herein. In this fourth embodiment, cells from a patient may contain unknown genetic mutations associated with the first disease.

The method of the fourth embodiment of the second aspect may further comprise testing one or more of a plurality of identified test agents on the cells from the patient having the first disease, wherein the identified test agents comprise test agents identified as modulating the phenotype morphological profile of the cultivated pathogenic transduced cells to a treated morphological profile that is similar to that of a control phenotype morphological profile. The test agents may comprise small molecules, biological compounds, or combinations thereof.

In this embodiment, the control phenotype morphological profile may be generated by morphologically profiling cultivated non-pathogenic transduced cells to develop a control phenotype morphological profile. Furthermore, the cultivated non-pathogenic transduced cells may be obtained by a method that comprises introducing Bacmam vectors not configured to overexpress one or more genes, underexpress one or more genes, or combinations of. Like an embodiment of the first aspect, introducing Bacman vectors not configured to overexpress or underexpress one or more genes or combinations thereof may comprise introducing one or more Bacmam vectors that have no genetic payload, carry nucleic acids encoding for a scrambled messenger ribonucleic acid, carry one or more genes but configured to localize the encoded protein incorrectly or to express a truncated protein, carry a non-pathogenic gene for overexpression, or combinations thereof. Concentrations of Bacmam vectors used to obtain pathogenic transduced cells and concentrations used to obtain non-pathogenic transduced cells may or may not be equal.

In a fifth embodiment of the second aspect, identifying the first disease phenotype morphological profile associated with the first disease comprises cultivating pathogenic transduced cells to overexpress one or more genes known to be associated with the first disease, underexpress one or more genes known to be associated with the first disease, or combinations thereof, to exhibit a disease phenotype in cultivated pathogenic transduced cells. The method further comprises identifying the phenotype morphological profiles as similar to the first disease phenotype morphological profile and identifying any genes overexpressed or underexpressed differently than in the cultivated pathogenic transduced cells, wherein any genes overexpressed or underexpressed differently than in the cultivated pathogenic transduced cells are additional genes associated with the first disease. In this embodiment, the method may further comprise prior to the cultivating steps obtaining pathogenic transduced cells that overexpress one or more genes known to be associated with the first disease, underexpress one or more genes known to be associated with the first disease, or combinations thereof comprising introducing Bacmam vectors containing the one or more genes known to be associated with the first disease and configured to transduce overexpression, underexpression, or combinations thereof of the one or more genes known to be associated with the first disease. Prior to the cultivating steps, the method may include obtaining non-pathogenic transduced cells that do not overexpress one or more genes known to be associated with the first disease, underexpress one or more genes known to be associated with the first disease, or combinations thereof.

The method of the fifth embodiment of the second aspect may further comprise testing one or more of a plurality of identified test agents on the cells from the patient having the first disease, wherein the identified test agents comprise test agents identified as modulating the phenotype morphological profile of the cultivated pathogenic transduced cells to a treated morphological profile that is similar to that of a control phenotype morphological profile. The test agents may comprise small molecules, biological compounds, or combinations thereof.

Also disclosed herein are high-throughput screening systems. In a third aspect are high-throughput screening systems where the system comprises a memory to store a plurality of phenotype morphological profiles and processor circuitry to receive a first set of image data for cultivated pathogenic transduced cells overexpressing a gene of interest associated with a disease, receive a second set of image data from cultivated non-pathogenic transduced cells not overexpressing the gene of interest, and receive a third set of image data from treated cultivated pathogenic transduced cells treated with one or more of a plurality of test agents, or combinations thereof. Further, the system comprises processor circuitry to generate a disease phenotype morphological profile from the first set of image data, generate a control phenotype morphological profile from the second set of image data, generate a treated phenotype morphological profile from the third set of image data for each of the one or more of the plurality of test agents or combinations thereof, and assign a similarity value to each of the treated phenotype morphological profiles relative to the control phenotype morphological profile.

In an embodiment of the third aspect, similar to some embodiments of the first and second aspects, generating any of the phenotype morphologically profiles may comprise tracking 100 or more cellular features, 500 or more cellular features, or 1000 or more cellular features. The cellular features may be determined and/or the outputs reduced using any of the methods disclosed herein.

In another embodiment of the third aspect, the system further comprises processor circuitry to determine a score for the treated morphological profile relative to the control phenotype morphological profile and to identify the treated morphological profile as similar to the control phenotype morphological profile if the score is within a selected range. In another embodiment, the system further comprises processor circuitry to determine a value for each morphological feature of a specific set of morphological features of the treated morphological profile relative to corresponding morphological features of the control phenotype morphological profile and identify the treated morphological profile as similar to the control phenotype morphological profile if the value for each morphological feature of the specific set is within a selected range. In another embodiment, the system further comprises processor circuitry to determine a value for each morphological feature of the treated morphological profile relative to corresponding morphological features of the control phenotype morphological profile and identify the treated morphological profile as similar to the control phenotype morphological profile if a selected percentage of the values are within a selected range. The selected range may be specific to the value for each morphological feature or each of the values may be scaled and the selected range may be a single selected range that applies to all of the scaled values.

In a fourth aspect, high-throughput screen systems comprise an interface to access a library of a plurality of phenotype morphological profiles and processor circuitry to access, via the interface, a library of a plurality of phenotype morphological profiles, wherein each of the plurality of phenotype morphological profiles were generated from cultivated pathogenic transduced cells that overexpressed one or more genes, underexpressed one or more genes, or combinations thereof. The systems further comprise processor circuitry to compare a first disease phenotype morphological profile with and identify any of the plurality of the phenotype morphological profiles that are similar to the first disease phenotype morphological profile.

In an embodiment of the fourth aspect, the systems further comprise processor circuitry to determine a score for the first disease phenotype morphological profile relative to each of the plurality of phenotype morphological profiles and identify the first disease phenotype morphological profile as similar to each of the plurality of phenotype morphological profiles if the score is within a selected range. Another embodiment of the systems of the fourth aspect includes systems that further comprise processor circuitry to determine a value for each morphological feature of a specific set of morphological features of the first disease phenotype morphological profile relative to corresponding morphological features of each of the plurality of phenotype morphological profiles and identify the first disease phenotype morphological profile as similar to each of the control plurality of phenotype morphological profiles if the value for each morphological feature of the specific set is within a selected range.

In another embodiment of the fourth aspect, the systems further comprise processor circuitry to determine a value for each morphological feature of the first disease phenotype morphological profile relative to corresponding morphological features of each of the plurality of phenotype morphological profiles and identify the first disease phenotype morphological profile as similar to each of the plurality of phenotype morphological profiles if a selected percentage of the values are within a selected range. Similar to some embodiments of various aspects, the selected range may be specific to the value for each morphological feature or each of the values may be scaled and the selected range may be a single selected range that applies to all of the scaled values.

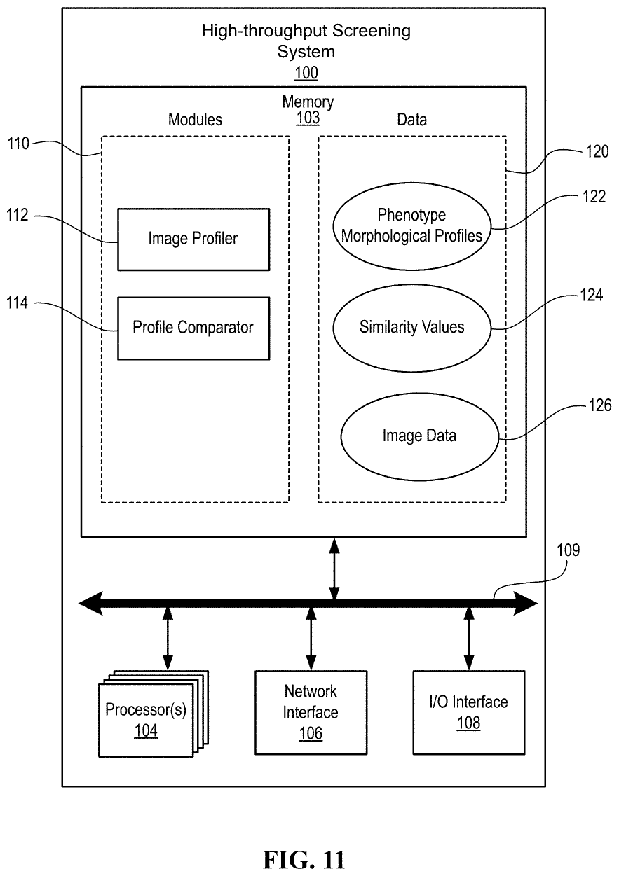

FIG. 11 is a block diagram of a high-throughput screening system 100 according to one embodiment. The high-throughput screening system 100 may perform the methods and use the techniques described with reference to the other figures in the specification. The high-throughput screening system 100 can include a memory 103, one or more processors 104, a network interface 106, an input/output interface 108, and a system bus 109.

The one or more processors 104 may include one or more general purpose devices, such as an Intel®, AMD®, or other standard microprocessor. The one or more processors 104 may include a special purpose processing device, such as ASIC, SoC, SiP, FPGA, PAL, PLA, FPLA, PLD, or other customized or programmable device. The one or more processors 104 can perform distributed (e.g., parallel) processing to execute or otherwise implement functionalities of the presently disclosed embodiments. The one or more processors 104 may run a standard operating system and perform standard operating system functions. It is recognized that any standard operating systems may be used, such as, for example, Microsoft® Windows®, Apple® MacOS®, Disk Operating System (DOS), UNIX, IRJX, Solaris, SunOS, FreeBSD, Linux®, ffiM® OS/2® operating systems, and so forth.

The memory 103 may include static RAM, dynamic RAM, flash memory, one or more flip-flops, ROM, CD-ROM, DVD, disk, tape, or magnetic, optical, or other computer storage medium. The memory 103 may include a plurality of program modules 110 and program data 120. The memory 103 may be local to the high-throughput screening system 100, as shown, or may be distributed and/or remote relative to the High-throughput screening system 100.

Data generated or used by the high-throughput screening system 100, such as by the program modules 110 or other modules, may be stored on the memory 103, for example, as stored program data 120. The data 120 may be organized as one or more databases.

The data 120 may include a phenotype morphological profiles 122, similarity values 124 and image data 126. The processor(s) 104 may access the data 120 using a memory interface. In some embodiments, the phenotype morphological profiles 122 may include a library of a plurality of phenotype morphological profiles, wherein each of the plurality of phenotype morphological profiles were generated from cultivated pathogenic transduced cells that overexpressed one or more genes, under expressed one or more genes, or combinations thereof.

In some embodiments, the image data 126 may include a first set of image data for cultivated pathogenic transduced cells overexpressing a gene of interest associated with a disease; a second set of image data from cultivated non-pathogenic transduced cells not overexpressing the gene of interest; and a third set of image data from treated cultivated pathogenic transduced cells treated with one or more of a plurality of test agents, or combinations thereof. The phenotype morphological profiles 122 may be generated using the image data 126.

In some embodiments, the similarity values 124 may be assigned to each of a set of treated phenotype morphological profiles relative to a control phenotype morphological profile. In some embodiments, the similarity values 124 may compare a plurality of the phenotype morphological profiles to a disease phenotype morphological profile.

The program modules 110 may include all or portions of other elements of the high-throughput screening system 100. The program modules 110 may run multiple operations concurrently or in parallel by or on the one or more processors 104. In some embodiments, portions of the disclosed modules, components, and/or facilities are embodied as executable instructions embodied in hardware or firmware, or stored on a non-transitory, machine-readable storage medium. The executable instructions may comprise computer program code that, when executed by a processor and/or computing device, cause a computing system to implement certain processing steps, procedures, and/or operations, as disclosed herein. The modules, components, and/or facilities disclosed herein may be implemented and/or embodied as a driver, a library, an interface, an API, FPGA configuration data, firmware (e.g., stored on an EEPROM), and/or the like. In some embodiments, portions of the modules, components, and/or facilities disclosed herein are embodied as machine components, such as general and/or application-specific devices, including, but not limited to: circuits, integrated circuits, processing components, interface components, hardware controller(s), storage controller(s), programmable hardware, FPGAs, ASICs, and/or the like. Accordingly, the modules disclosed herein may be referred to as controllers, layers, services, engines, facilities, drivers, circuits, subsystems, and/or the like.

The modules 110 may comprise an image profiler 112 and a profile comparator 114. The image profiler 122 may use the image data 126 to generate the phenotype morphological profiles 122 using techniques discussed herein. The profile comparator 114 may compare and assign the similarity values 124 to the phenotype morphological profiles 122 using techniques discussed herein.

The input/output interface 108 may facilitate user interaction with one or more input devices and/or one or more output devices. The input device(s) may include a keyboard, mouse, touchscreen, light pen, tablet, microphone, sensor, or other hardware with accompanying firmware and/or software. The output device(s) may include a monitor or other display, printer, speech or text synthesizer, switch, signal line, or other hardware with accompanying firmware and/or software. For example, in one embodiment, the input/output interface 108 comprises a display to provide a graphical user interface (GUI) illustrating the potential ablation perimeters. The input/output interface 108 can receive the user input data. In some embodiments, the input/output interface 108 is a touchscreen, and the size input is received via the touchscreen. In some embodiments, the input/output interface 108 can superimpose the target ablation perimeters on an image of the tissue.

The network interface 106 may facilitate communication with other computing devices and/or networks and/or other computing and/or communications networks. The network interface 106 may be equipped with conventional network connectivity, such as, for example, Ethernet (IEEE 1102.3), Token Ring (IEEE 1102.5), Fiber Distributed Datalink Interface (FDDI), or Asynchronous Transfer Mode (ATM). Further, the network interface 106 may be configured to support a variety of network protocols such as, for example, Internet Protocol (IP), Transfer Control Protocol (TCP), Network File System over UDP/TCP, Server Message Block (SMB), Microsoft® Common Internet File System (CIFS), Hypertext Transfer Protocols (HTTP), Direct Access File System (DAFS), File Transfer Protocol (FTP), Real-Time Publish Subscribe (RTPS), Open Systems Interconnection (OSI) protocols, Simple Mail Transfer Protocol (SMTP), Secure Shell (SSH), Secure Socket Layer (SSL), and so forth.

The system bus 109 may facilitate communication and/or interaction between the other components of the High-throughput screening system 500, including the one or more processors 104, the memory 103, the input/output interface 108, and the network interface 106.

In some cases, well-known features, structures or operations are not shown or described in detail. Furthermore, the described features, structures, or operations may be combined in any suitable manner in one or more embodiments. It will also be readily understood that the components of the embodiments as generally described and illustrated in the figures herein could be arranged and designed in a wide variety of different configurations.

Several aspects of the embodiments described may be implemented as software modules or components. As used herein, a software module or component may include any type of computer instruction or computer executable code located within a memory device and/or transmitted as electronic signals over a system bus or wired or wireless network. A software module or component may, for instance, comprise one or more physical or logical blocks of computer instructions, which may be organized as a routine, program, object, component, data structure, etc., that performs one or more tasks or implements particular abstract data types.

In certain embodiments, a particular software module or component may comprise disparate instructions stored in different locations of a memory device, which together implement the described functionality of the module. Indeed, a module or component may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software modules or components may be located in local and/or remote memory storage devices. In addition, data being tied or rendered together in a database record may be resident in the same memory device, or across several memory devices, and may be linked together in fields of a record in a database across a network.

Embodiments may be provided as a computer program product including a non-transitory computer and/or machine-readable medium having stored thereon instructions that may be used to program a computer (or other electronic device) to perform processes described herein. For example, a non-transitory computer-readable medium may store instructions that, when executed by a processor of a computer system, cause the processor to perform certain methods disclosed herein. The non-transitory computer-readable medium may include, but is not limited to, hard drives, floppy diskettes, optical disks, CD-ROMs, DVD-ROMs, ROMs, RAMs, EPROMs, EEPROMs, magnetic or optical cards, solid-state memory devices, or other types of machine-readable media suitable for storing electronic and/or processor executable instructions.

### EXAMPLES

### Example 1—Vector Constructs for Transducing Cells (and Setting up for Morphological Profiling)

Genes of interest were inserted into BacMam vectors (Montana Molecular) to generate BacMam constructs for transducing cells using known methods.

Cells were transduced with BacMam to overexpress BRAF V600E nonsense or BRAF V600E FLAG then immunostained. Immunofluorescent staining of the C-terminal FLAG tag on BRAF V600E (FIG. 1 left panel) and BRAF V600E nonsense (FIG. 1 right panel) are shown. Nuclei are shown in red, the FLAG tag is shown in green. The stop codons in the nonsense mutant did not prevent expression of the FLAG tagged protein.

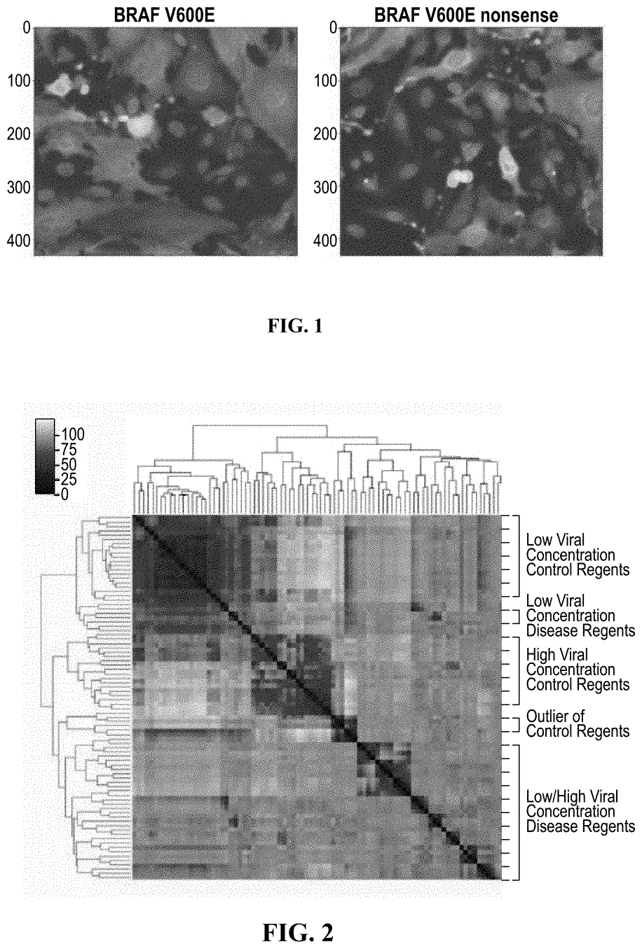

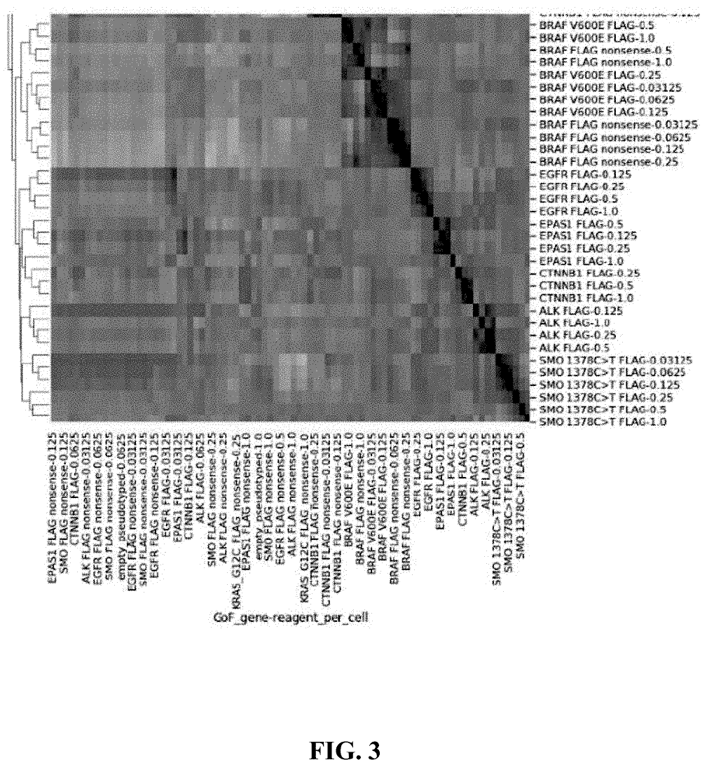

Unsupervised clustering of cosine similarity between pairs of reagents in BacMam titration experiment was determined. Major cluster on top left is most viruses at low doses, independent of viral payload. Center cluster is empty viruses at higher doses, suggesting a basal infection phenotype. Collection of smaller clusters at bottom right shows grouping of overexpression phenotypes at higher doses (FIG. 2). Clustering of disease reagents in higher doses is shown (FIG. 3, a zoom-in image of FIG. 2).

### Example 2—Collecting Features for Morphologically Profiling Cells

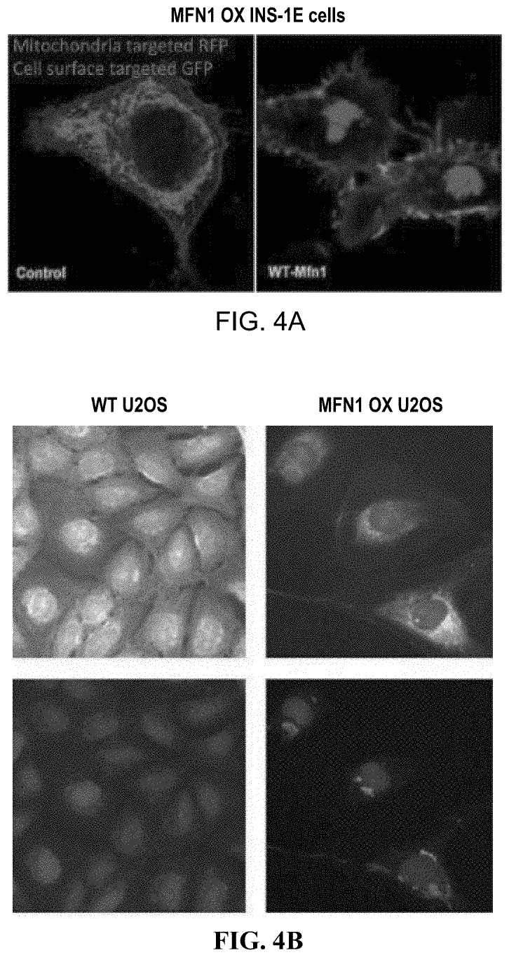

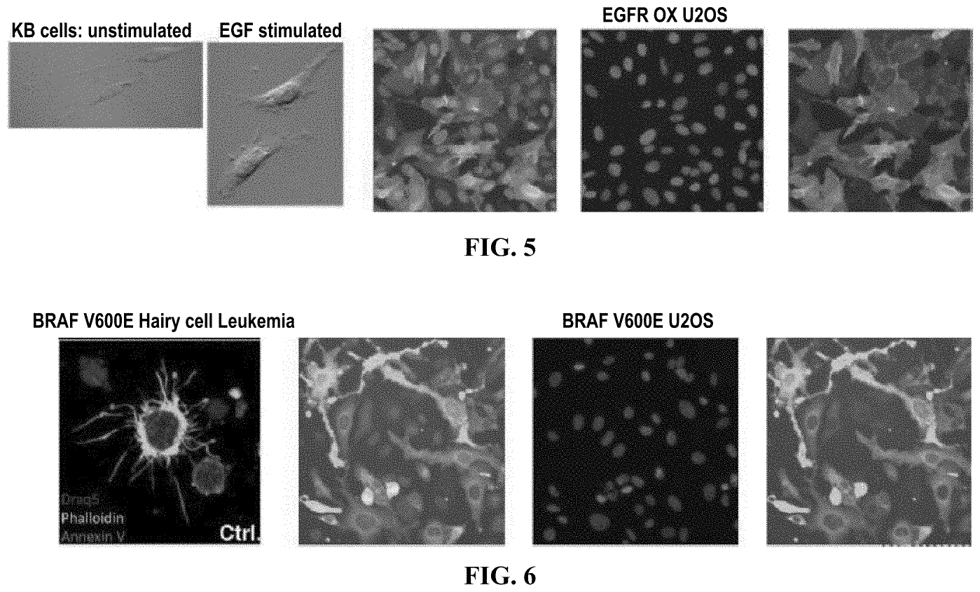

U2OS cells were transduced with BacMam carrying MFN1, EGFR, or BRAF, then stained and imaged. Overexpression of MFN1 show mitochondrial clustering (FIG. 4B) in a similar manner as the mitochondrial clustering in MFN1-overexpressing cells reported by others (FIG. 4A, Park et al. Korean J Physiol Pharmacol. 2012 Feb.; 16(1): 71-77 which is incorporated herein by reference in its entirety). A ruffled edge phenotype is shown. U2OS cells were transduced with BacMam carrying EGFR-FLAG then stained for DNA (red) and EGFR-FLAG (green) (FIG. 5 panels on far right, second from right, and center). Overexpression of EGFR in cells shows a ruffled phenotype, similar to EGF stimulation reported by others (FIG. 5 panels on far left and second from left, Bretscher et al. Current Biology Volume 8, Issue 12, 4 Jun. 1998, Pages 721-724, S1-S4 which is incorporated herein by reference in its entirety. In another example, U2OS cells that were transduced with BacMam carrying V600E-FLAG were stained then imaged (FIG. 6 second, third, and fourth panels from the left). Similar to that seen and reported by others in hairy cell leukemia (FIG. 6 left panel, Falini et al. Blood. 2016;128(15):1918-1927 which is incorporated herein by reference in its entirety), U2OS cells overexpressing V600E show formation of filaments.

### Example 3—Morphological Profiling and Comparison of Profiles Between Pathogenic and Non-Pathogenic Transduced Cells

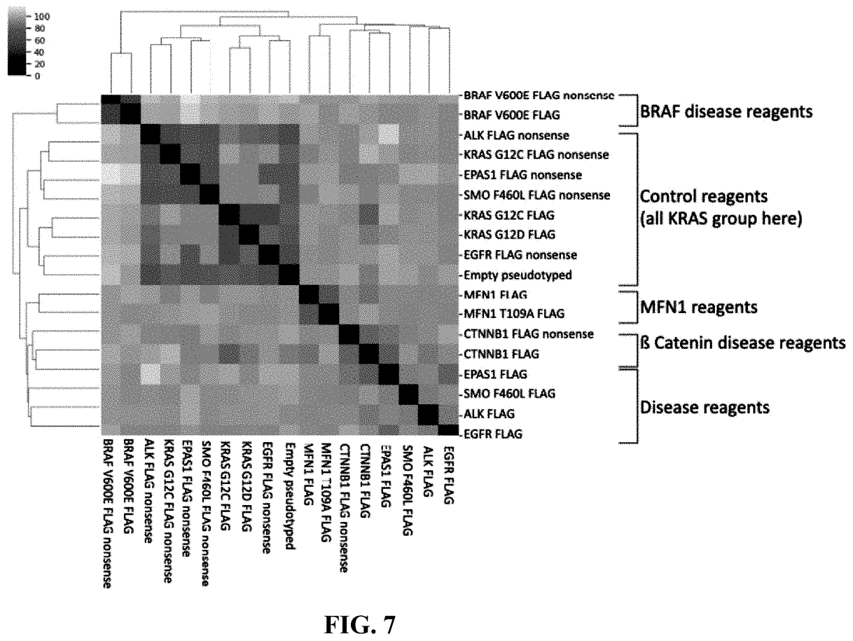

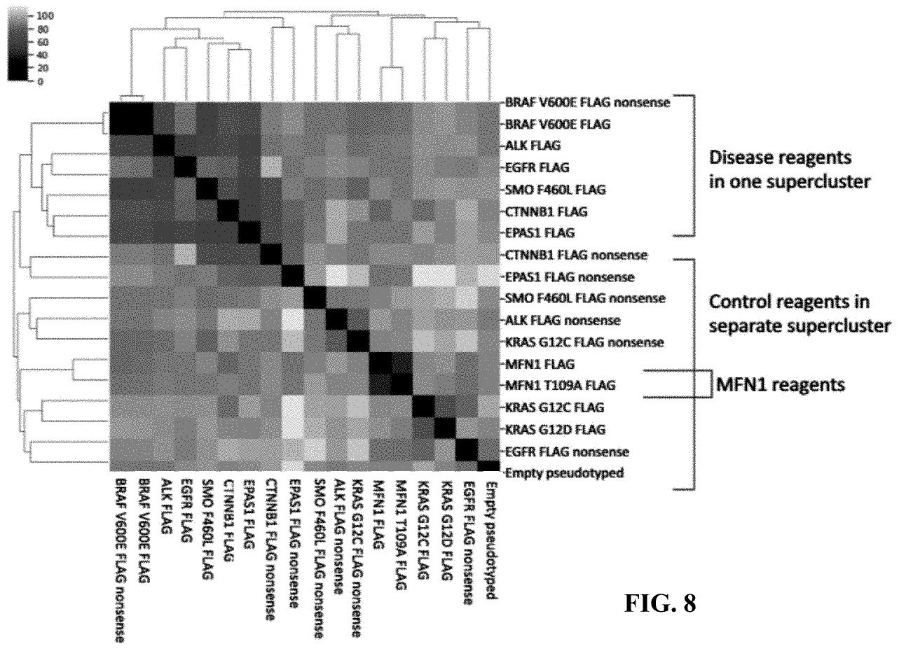

Pathogenic and non-pathogenic transduced cells were cultured then morphologically profiled. A heatmap of cosine similarities between all pairs of groups (or reagents), origined by method one were generated (FIG. 7). Shown are cells transduced with BacMam vectors having BRAF V600E, KRAS G12C, KRAS G12D, MFN1, MFN1T109A, CTNNB1B, EPAS1, SMO F460L, ALK, EGFR, and some of their nonsense counterparts. A large block of similarity largely consisting of BacMams expressing nonsense transcripts but also including pseudotyped empty BacMams and BacMams expressing KRAS variants that translate into protein but do not function was observed (FIG. 7). Cosine similarities between all pairs of feature vectors that were origin by method two in FIG. 7 (FIG. 8). Note similarity between BRAF V600E and BRAF V600E nonsense, suggesting that they share a near-identical phenotype. Furthermore, pathogenic or disease reagents and non-pathogenic or control reagents clustered separately.

Phenotype scoring was conducted for cells overexpressing various genes (Table 1). Cross-validation (CV) angle was calculated using a process where each guide from a sample was compared to the rest of the sample. The CV angle is an average of angles for all individual guides belonging to the gene, wherein the angle is between a selected guide vector and the average vector for the rest of the guides belonging to the same gene. The p-value is a probability of getting a CV angle smaller than CV-angle of interest by comparing random guides that do not belong to the same gene by using the same cross-validation procedure. For example, 6 random guides are selected then each guide are compared to the average of the remaining 5 randomly selected guides. The average of the resulting angles are calculated. To calculate z factors, the following equation from Zhang et al. J Biomol Screen. 1999; 4(2):67-73 was used:

$$Z = 1 - \frac{3 {σ}_{s} + 3 {σ}_{c}}{\vert {µ}_{s} - {µ}_{c} \vert}$$

In the equation above, σₛ is the standard deviation of healthy group, σ_{c} is the standard deviation of diseased group, μₛ is the average of the health group, and μ_{c} is the average of the diseased group. Zhang et al. J Biomol Screen. 1999; 4(2):67-73 is incorporated by reference herein in its entirety.

The results suggest that various disease modeling BacMam reagents tested produce strong phenotypes that are distinct from control groups.

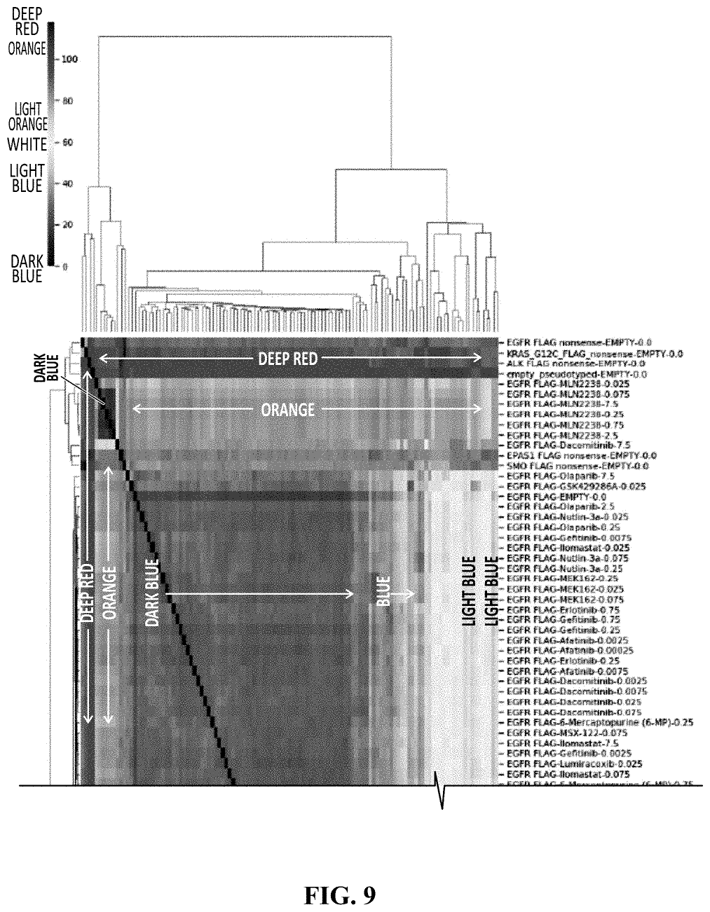

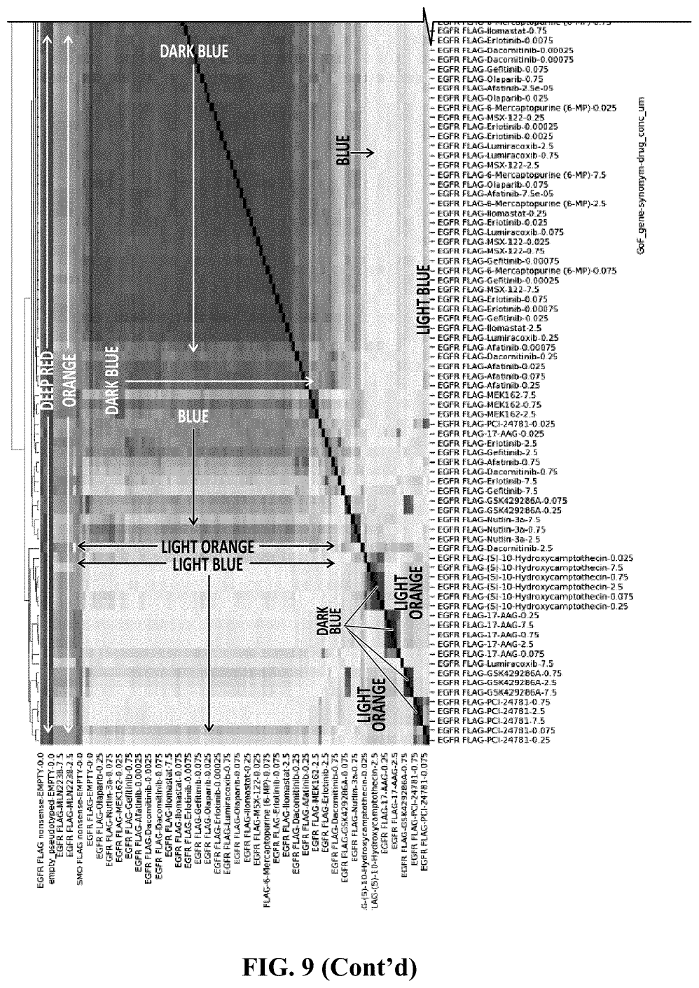

Cells transduced to overexpress EGFR and cells not transduced to overexpress EGFR were morphologically profiled and evaluated (FIG. 9).

TABLE 1
Phenotype scores of transduced cells

| Overexpressed gene | CV angle | CV angle pval | Z factor |
|---|---|---|---|
| BRAF V600E | 30.9 | 0 | 0.845 |
| MFN1 T109A | 31.8 | 0 | 0.650 |
| ALK | 32.2 | 0 | 0.555 |
| BRAF V600E | 34.0 | 0 | 0.806 |
| nonsense | | | |
| SMO F460L | 36.7 | 0 | 0.797 |
| EGFR | 47.3 | 0 | 0.559 |
| MFN1 | 48.3 | 0 | 0.639 |
| EPAS1 | 52.7 | 0 | 0.666 |
| CTNNB1 nonsense | 53.4 | 0 | 0.564 |
| CTNNB1 | 70.3 | 0 | 0.459 |
| SMO F460L nonsense | 84.9 | 0.487 | −0.385 |
| KRAS G12D | 86.1 | 0.659 | 0.198 |
| EGFR nonsense | 86.2 | 0.673 | 0.139 |
| KRAS G12C | 86.9 | 0.776 | 0.008 |
| ALK nonsense | 87.2 | 0.811 | −0.737 |
| Empty pseudotyped | 88.1 | 0.896 | −0.312 |
| EPAS1 nonsense | 89.5 | 0.969 | −0.229 |

### Example 4—Identification of Test Agents That Modulate Phenotypes

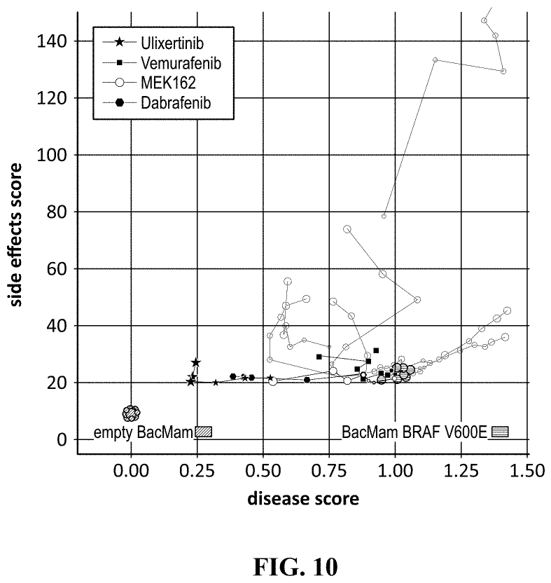

Cells transduced with BacMam BRAF V600E were treated with varying concentrations of 18 compounds which include dabrafenib and vemurafenib, which target BRAF, and ulixertinib and MEK162, which target downstream biology. Separation from the funnel shown for dabrafenib, ulixertinib, and MEK162 (FIG. 10). The difference in features caused by transducing the cells with BRAF V600E was determined. The effect of compounds on those features was determined within the BRAF V600E-transduced cells and also the effects of compounds on features not related to transduction with BRAF V600E. Most compounds had a more noticable effect on features not attributable to BRAF V600E than they do on features attributable to BRAF V600E, as illustrated in FIG. 10. Vemurafenib is at the outer edge of the funnel. The efficacy of test agents in reversing a disease state can be quantified. This is calculated based on morphological profiling of cells treated with a test agent compared to cells not treated with a test agent. Ulixertinib, dabrafenib, MEK162, and vemurafenib are also shown to be some of the top test agents when reveral of the disease state is quantified in this way. These results suggest rescue of BRAF V600E overexpression phenotype.

Test agents were also used as a treatment on EGFR-overexpressing cells. Cells were first transduced with BacMam EGFR then treated with a collection of compounds including afatinib, gefitinib, dacomitinib, and erlotinib. Heatmaps of cosine similarity can be generated that compare, for example, not-treated EGFR overexpressing cells versus each group having EGFR overexpressing cells group treated with a test agent (FIG. 9). Quantification of the reversal of the disease state suggested that afatinib and dacomitinib were the top two test agents that can modulate the phenotypes of EGFR overexpressing cells.

### Example 5—Morphological Profile Comparison Between Cells Associated With a Disease and a Library of Morphological Profiles

Cells from a patient are morphologically profiled then compared to a library of morphological profiles. The library includes morphological profiles of cells having overexpression, underexpression, or a combination of overexpression and underexpression, of genes associated with a disease or a particular morphological phenotype. The morphologically profiled cells from the patient are evaluated for similarity to one or more morphological profiles in the library. If morphologically similar, then the cells from the patient may have overexpression, underexpression, or a combination of overexpression and underexpression, of particular genes indicated by the matched profiles in the library. Further testing can be performed to confirm overexpression, underexpression, or a combination of overexpression and underexpression, of particular genes indicated by the matched profiles in the library.

It will be apparent to those having skill in the art that many changes may be made to the details of the above-described embodiments without departing from the underlying principles of the invention.

1. A method of high-throughput screening test agents for treating a disease, the method comprising:
cultivating pathogenic transduced cells overexpressing a gene of interest associated with a disease to exhibit a disease phenotype in cultivated pathogenic transduced cells;
cultivating non-pathogenic transduced cells not overexpressing the gene of interest to exhibit a control phenotype in cultivated non-pathogenic transduced cells;
morphologically profiling the cultivated pathogenic transduced cells to develop a disease phenotype morphological profile;
morphologically profiling the cultivated non-pathogenic transduced cells to develop a control phenotype morphological profile;
adding one or more of a plurality of test agents, or combinations thereof, to the cultivated pathogenic transduced cells to form treated cultivated pathogenic transduced cells;
morphologically profiling the treated cultivated pathogenic transduced cells; and
identifying any of the plurality of the test agents, or combinations thereof, that modulate the disease phenotype morphological profile of the treated cultivated pathogenic transduced cells to a treated morphological profile that is similar to that of the control phenotype morphological profile.

2. The method of claim 1, wherein the test agents comprise small molecules, biological compounds, or combinations thereof.

3. The method of claim 1, wherein the morphologically profiling in any of the steps utilizes imaging the relevant cells.

4. The method of claim 1, wherein the morphologically profiling in any of the steps comprises tracking:
staining intensities in one or more imaging channels;
correlations between imaging channels;
textural patterns;
size and shape of cellular structures;
geometric relationships between intracellular structures;
geometric relationships between adjacent cells; or combinations thereof.

5. The method of claim 1, wherein the morphologically profiling in any of the steps comprises tracking 100 or more cellular features, 500 or more cellular features, or 1000 or more cellular features.

6. The method of claim 5, wherein the cellular features are determined using deep learning training sets, hand-tuned feature extraction, or combinations thereof.

7. The method of claim 5, wherein the cellular features are determined at least in part by imaging cells with or without fluorescent or non-fluorescent stains or combinations thereof.

8. The method of claim 5, wherein the morphological profiling comprises reducing the output of the cellular features using principal component analysis.

9. The method of claim 1, wherein obtaining pathogenic transduced cells overexpressing the gene of interest comprises introducing Bacmam vectors containing the gene of interest and configured to transduce overexpression of the gene of interest.

10. The method of claim 1, further comprising prior to the cultivating steps obtaining pathogenic transduced cells overexpressing a gene of interest associated with a gain-of-function disease and obtaining non-pathogenic transduced cells not overexpressing the gene of interest.

11. The method of claim 10, wherein obtaining non-pathogenic transduced cells not overexpressing the gene of interest comprises introducing Bacmam vectors not configured to overexpress the gene of interest.

12. The method of claim 11, wherein introducing Bacmam vectors not configured to overexpress the gene of interest comprises introducing one or more Bacmam vectors:
having no genetic payload;
carrying nucleic acids encoding for a scrambled messenger ribonucleic acid;
carrying the gene of interest but configured to localize the encoded protein incorrectly or to express a truncated protein;
carrying a non-pathogenic gene for overexpression; or combinations thereof.

13. The method of claim 11, wherein a concentration of the Bacmam vectors containing the gene of interest is the same as a concentration of Bacmam vectors not configured to overexpress the gene of interest.

14. The method of claim 1, wherein the gene of interest comprises a first gene of interest and wherein cultivating the pathogenic transduced cells to exhibit a disease phenotype in cultivated pathogenic transduced cells comprises cultivating a first pathogenic transduced cells to exhibit a first disease phenotype in a first cultivated pathogenic transduced cells grown in a first reservoir of a cell culture vessel.

15. The method of claim 14, wherein the gene of interest comprises a second gene of interest and wherein cultivating the pathogenic transduced cells to exhibit a disease phenotype in cultivated pathogenic transduced cells comprises cultivating a second pathogenic transduced cells to exhibit a second disease phenotype in a second cultivated pathogenic transduced cells grown in a second well of the well plate.

16. The method of claim 15, wherein the first well contains a first pre-selected initial quantity of cells, wherein the second well contains a second pre-selected initial quantity of cells, wherein the first pre-selected initial quantity is different from the second pre-selected initial quantity of cells, and wherein after the cultivating step, a quantity of cells in the first well is approximately the same as a quantity of cells in the second well.

17. The method of claim 10, wherein obtaining pathogenic transduced cells overexpressing a gene of interest and obtaining non-pathogenic transduced cells not overexpressing the gene of interest comprises transducing the cells in individual wells of the well plate.

18. The method of claim 1, further comprising determining a score for the treated morphological profile relative to the control phenotype morphological profile and identifying the treated morphological profile as similar to the control phenotype morphological profile if the score is within a selected range.

19. The method of claim 1, further comprising determining a value for each morphological feature of a specific set of morphological features of the treated morphological profile relative to corresponding morphological features of the control phenotype morphological profile and identifying the treated morphological profile as similar to the control phenotype morphological profile if the value for each morphological feature of the specific set is within a selected range.

20. The method of claim 19, wherein the selected range is specific to the value for each morphological feature or wherein each of the values is scaled and the selected range is a single selected range that applies to all of the scaled values.

21. The method of claim 1, further comprising determining a value for each morphological feature of the treated morphological profile relative to corresponding morphological features of the control phenotype morphological profile and identifying the treated morphological profile as similar to the control phenotype morphological profile if a selected percentage of the values are within a selected range.

22. The method of claim 21, wherein the selected range is specific to the value for each morphological feature or wherein each of the values is scaled and the selected range is a single selected range that applies to all of the scaled values.

23. The method of claim 1, wherein the cultivated pathogenic transduced cells overexpressing the gene of interest associated with the disease also overexpress one or more additional genes associated with the disease, underexpress one or more genes associated with the disease, or combinations thereof.

24. A method of high-throughput screening diseases, the method comprising:
providing a first disease phenotype morphological profile associated with a first disease;
comparing the first disease phenotype morphological profile with a library of a plurality of phenotype morphological profiles, wherein each of the plurality of phenotype morphological profiles were generated from cultivated pathogenic transduced cells that overexpressed one or more genes, underexpressed one or more genes, or combinations thereof; and
identifying any of the plurality of the phenotype morphological profiles that are similar to the first disease phenotype morphological profile.

25. The method of claim 24, further comprising determining a score for the first disease phenotype morphological profile relative to each of the plurality of phenotype morphological profiles and identifying the first disease phenotype morphological profile as similar to each of the plurality of phenotype morphological profiles if the score is within a selected range.

26. The method of claim 24, further comprising determining a value for each morphological feature of a specific set of morphological features of the first disease phenotype morphological profile relative to corresponding morphological features of each of the plurality of phenotype morphological profiles and identifying the first disease phenotype morphological profile as similar to each of the plurality of phenotype morphological profiles if the value for each morphological feature of the specific set is within a selected range.

27. The method of claim 26, wherein the selected range is specific to the value for each morphological feature or wherein each of the values is scaled and the selected range is a single selected range that applies to all of the scaled values.

28. The method of claim 24, further comprising determining a value for each morphological feature of the first disease phenotype morphological profile relative to corresponding morphological features of each of the plurality of phenotype morphological profiles and identifying the first disease phenotype morphological profile as similar to each of the plurality of phenotype morphological profiles if a selected percentage of the values are within a selected range.

29. The method of claim 28, wherein the selected range is specific to the value for each morphological feature or wherein each of the values is scaled and the selected range is a single selected range that applies to all of the scaled values.

30. The method of claim 24, wherein providing the first disease phenotype morphological profile associated with the first disease comprises morphologically profiling cells from a patient having the first disease.

31. The method of claim 30, wherein the morphologically profiling in any of the steps utilizes imaging the relevant cells.

32. The method of claim 30, wherein the morphologically profiling in any of the steps comprises tracking:
staining intensities in one or more imaging channels;
correlations between imaging channels;
textural patterns;
size and shape of cellular structures;
geometric relationships between intracellular structures;
geometric relationships between adjacent cells; or
combinations thereof.

33. The method of claim 30, wherein the morphologically profiling in any of the steps comprises tracking 100 or more cellular features, 500 or more cellular features, or 1000 or more cellular features.

34. The method of claim 33, wherein the cellular features are determined using deep learning training sets.

35. The method of claim 33, wherein the cellular features are determined using 6 fluorescent stains, or combinations thereof.

36. The method of claim 33, wherein the morphological profiling comprises reducing the output of the cellular features using principal component analysis.

37. The method of claim 30, wherein the cells from the patient contain unknown genetic mutations associated with the first disease.

38. The method of claim 30, further comprising testing one or more of a plurality of identified test agents on the cells from the patient having the first disease, wherein the identified test agents comprise test agents identified as modulating the phenotype morphological profile of the cultivated pathogenic transduced cells to a treated morphological profile that is similar to that of a control phenotype morphological profile.

39. The method of claim 38, wherein the test agents comprise small molecules, biological compounds, or combinations thereof.

40. The method of claim 38, wherein the control phenotype morphological profile is generated by morphologically profiling cultivated non-pathogenic transduced cells to develop a control phenotype morphological profile.

41. The method of claim 40, wherein the cultivated non-pathogenic transduced cells are obtained by a method that comprises introducing Bacmam vectors not configured to overexpress one or more genes, underexpress one or more genes, or combinations thereof.

42. The method of claim 41, wherein introducing Bacmam vectors not configured to overexpress one or more genes, underexpress one or more genes, or combinations thereof comprises introducing one or more Bacmam vectors:
having no genetic payload;
carrying nucleic acids encoding for a scrambled messenger ribonucleic acid;
carrying one or more genes but configured to localize the encoded protein incorrectly or to express a truncated protein;
carrying a non-pathogenic gene for overexpression; or
combinations thereof.

43. The method of claim 41, wherein a concentration of the Bacmam vectors containing one or more genes is the same as a concentration of Bacmam vectors not configured to overexpress one or more genes, underexpress one or more genes, or combinations thereof.

44. The method of claim 24, wherein providing the first disease phenotype morphological profile associated with the first disease comprises cultivating pathogenic transduced cells to overexpress one or more genes known to be associated with the first disease, underexpress one or more genes known to be associated with the first disease, or combinations thereof, to exhibit a disease phenotype in cultivated pathogenic transduced cells; and further comprising for the phenotype morphological profiles identified as similar to the first disease phenotype morphological profile, identifying any genes overexpressed or underexpressed differently than in the cultivated pathogenic transduced cells, wherein any genes overexpressed or underexpressed differently than in the cultivated pathogenic transduced cells are additional genes associated with the first disease.

45. The method of claim 44, further comprising prior to the cultivating steps obtaining pathogenic transduced cells that overexpress one or more genes known to be associated with the first disease, underexpress one or more genes known to be associated with the first disease, or combinations thereof comprise introducing Bacmam vectors containing the one or more genes known to be associated with the first disease and configured to transduce overexpression, underexpression, or combinations thereof of the one or more genes known to be associated with the first disease.

46. The method of claim 44, further comprising prior to the cultivating steps obtaining pathogenic transduced cells that overexpress one or more genes known to be associated with the first disease, underexpress one or more genes known to be associated with the first disease, or combinations thereof and obtaining non-pathogenic transduced cells that do not overexpress one or more genes known to be associated with the first disease, underexpress one or more genes known to be associated with the first disease, or combinations thereof.

47. The method of claim 44, further comprising testing one or more of a plurality of identified test agents on the cells from the patient having the first disease, wherein the identified test agents comprise test agents identified as modulating the phenotype morphological profile of the cultivated pathogenic transduced cells to a treated morphological profile that is similar to that of a control phenotype morphological profile.

48. The method of claim 47, wherein the test agents comprise small molecules, biological compounds, or combinations thereof.

49. A high-throughput screening system, the system comprising:
a memory to store a plurality of phenotype morphological profiles; and processor circuitry to:
receive a first set of image data for cultivated pathogenic transduced cells overexpressing a gene of interest associated with a disease;
receive a second set of image data from cultivated non-pathogenic transduced cells not overexpressing the gene of interest;
receive a third set of image data from treated cultivated pathogenic transduced cells treated with one or more of a plurality of test agents, or combinations thereof;
generate a disease phenotype morphological profile from the first set of image data;
generate a control phenotype morphological profile from the second set of image data;
generate a treated phenotype morphological profile from the third set of image data for each of the one or more of the plurality of test agents, or combinations thereof; and
assign a similarity value to each of the treated phenotype morphological profiles relative to the control phenotype morphological profile.

50. The system of claim 49, wherein generating any of the phenotype morphological profiles comprise tracking 100 or more cellular features, 500 or more cellular features, or 1000 or more cellular features.

51. The system of claim 50, wherein the cellular features are determined using deep learning training sets, hand-tuned feature extraction, or combinations thereof.

52. The system of claim 50, wherein the cellular features are determined at least in part by imaging cells with or without fluorescent or non-fluorescent stains or combinations thereof.

53. The system of claim 50, wherein generating any of the phenotype morphological profiles comprise reducing the output of the cellular features using principal component analysis.

54. The system of claim 49, wherein the system further comprises processor circuitry to determine a score for the treated morphological profile relative to the control phenotype morphological profile and identify the treated morphological profile as similar to the control phenotype morphological profile if the score is within a selected range.

55. The system of claim 49, wherein the system further comprises processor circuitry to determine a value for each morphological feature of a specific set of morphological features of the treated morphological profile relative to corresponding morphological features of the control phenotype morphological profile and identify the treated morphological profile as similar to the control phenotype morphological profile if the value for each morphological feature of the specific set is within a selected range.

56. The system of claim 55, wherein the selected range is specific to the value for each morphological feature or wherein each of the values is scaled and the selected range is a single selected range that applies to all of the scaled values.

57. The system of claim 49, wherein the system further comprises processor circuitry to determine a value for each morphological feature of the treated morphological profile relative to corresponding morphological features of the control phenotype morphological profile and identify the treated morphological profile as similar to the control phenotype morphological profile if a selected percentage of the values are within a selected range.

58. The system of claim 57, wherein the selected range is specific to the value for each morphological feature or wherein each of the values is scaled and the selected range is a single selected range that applies to all of the scaled values.

59. A high-throughput screening system, the system comprising:
an interface to access a library of a plurality of phenotype morphological profiles; and
processor circuitry to:
access, via the interface, a library of a plurality of phenotype morphological profiles, wherein each of the plurality of phenotype morphological profiles were generated from cultivated pathogenic transduced cells that overexpressed one or more genes, underexpressed one or more genes, or combinations thereof;
compare a first disease phenotype morphological profile with; and
identify any of the plurality of the phenotype morphological profiles that are similar to the first disease phenotype morphological profile.

60. The system of claim 59, wherein the system further comprises processor circuitry to determine a score for the first disease phenotype morphological profile relative to each of the plurality of phenotype morphological profiles and identify the first disease phenotype morphological profile as similar to each of the plurality of phenotype morphological profiles if the score is within a selected range.

61. The system of claim 59, wherein the system further comprises processor circuitry to determine a value for each morphological feature of a specific set of morphological features of the first disease phenotype morphological profile relative to corresponding morphological features of each of the plurality of phenotype morphological profiles and identify the first disease phenotype morphological profile as similar to each of the plurality of phenotype morphological profiles if the value for each morphological feature of the specific set is within a selected range.

62. The system of claim 61, wherein the selected range is specific to the value for each morphological feature or wherein each of the values is scaled and the selected range is a single selected range that applies to all of the scaled values.

63. The system of claim 59, wherein the system further comprises processor circuitry to determine a value for each morphological feature of the first disease phenotype morphological profile relative to corresponding morphological features of each of the plurality of phenotype morphological profiles and identify the first disease phenotype morphological profile as similar to each of the plurality of phenotype morphological profiles if a selected percentage of the values are within a selected range.

64. The system of claim 63, wherein the selected range is specific to the value for each morphological feature or wherein each of the values is scaled and the selected range is a single selected range that applies to all of the scaled values.

65. A method of high-throughput screening test agents for treating a disease, the method comprising:
cultivating pathogenic transduced cells overexpressing a gene of interest associated with a disease to exhibit a disease phenotype in cultivated pathogenic transduced cells;
cultivating non-pathogenic transduced cells not overexpressing the gene of interest to exhibit a control phenotype in cultivated non-pathogenic transduced cells;
profiling the cultivated pathogenic transduced cells to develop a disease phenotype profile;
profiling the cultivated non-pathogenic transduced cells to develop a control phenotype profile;
adding one or more of a plurality of test agents, or combinations thereof, to the cultivated pathogenic transduced cells to form treated cultivated pathogenic transduced cells;
profiling the treated cultivated pathogenic transduced cells; and
identifying any of the plurality of the test agents, or combinations thereof, that modulate the disease phenotype profile of the treated cultivated pathogenic transduced cells to a treated profile that is similar to that of the control phenotype profile.
